(19) United States
(12) Patent Application Publication
(10) Pub. No.: US 2007/0055250 A1
(43) Pub. Date: Mar. 8, 2007

(54) Cervical plates with spacer mechanism

(76) Inventor: Kamran Aflatoon, Newport Beach, CA (US)

Correspondence Address: Ober, Kaler, Grimes & Shriver, 120 East Baltimore Street, Baltimore, MD, 21202-1643, US

(21) Appl. No.: 11/484,357

(22) Filed: Jul. 11, 2006

Related U.S. Application Data
(60) Provisional application No. 60/698,232, filed on Jul. 11, 2005.

(51) Int. Cl. A61F 2/30

(52) U.S. Cl. 606/69

(57) ABSTRACT

The present invention is a cervical plate connector system comprising cervical plates and a spacer mechanism. Ridged rails span the entire midline of the plates. Screw holes are cut into the center of one side of each of the two plates. Screws are inserted into the screw holes of each of the plates, which attach to both the superior and inferior vertebral bodies of the cervical spine. The midline of the plates have ridged rails running between screw holes. A spacer mechanism connects to the ridged rails of plates, and moves along them until the healing is complete and the two plates are adjacent to each other. A locking mechanism locks the screws into the plates of the connector system without detracting from the mobility of the spacer mechanism. The locking mechanism comprises a locking plate having screw holes, and at least one plate cover.

### CROSS-REFERENCE TO RELATED APPLICATIONS

The present application derives priority from provisional patent application No. 60/698,232, filed Jul. 11, 2005.

### BACKGROUND OF THE INVENTION

1. Field of the Invention

The present invention relates to a cervical plate connector system for treatment of cervical spine conditions and more particularly to cervical plates with a spacer mechanism for use in a procedure called anterior cervical discectomy.

2. Description of the Background

If surgery is needed to alleviate nerve or spinal cord compression in a patient, then a surgeon may perform a procedure called anterior cervical discectomy. Typically with this procedure, the surgeon makes a small incision in the front of the neck to reach the upper part of the spine, the cervical spine, which is made up of seven vertebrae. Next, the surgeon retracts tissues and muscles (to reveal the level in the cervical spine) and then removes the disc and fills the space with bone graft. Finally, the surgeon screws in a cervical plate to the superior and inferior vertebral bodies, which stabilizes the cervical spine thereby allowing spinal fusion and healing. A variety of designs for cervical plate connector systems have been proposed over the years and include the following.

U.S. Pat. No. 6,780,186 to Errico et al. (Third Millennium Engineering LLC) issued Aug. 24, 2004 shows an anterior cervical plate having polyaxial locking screws and sliding coupling elements. The bone plate has elongated tapered holes in the top and bottom thereof, into which adjustable coupling elements are inserted. Screws are inserted through the coupling elements. The coupling elements are initially disposed in the holes in the plate such that they may slide axially therein. Once the screw head is fully seated in the coupling element, advancement of the screw causes the coupling element to crush lock to the plate and to the head of the screw.

U.S. Pat. No. 6,695,846 to Richelsoph et al. issued Feb. 24, 2004 shows a bone plate and screw retaining mechanism with a snap ring for placement into an aperture of a base plate and movable on the plate between locked and unlocked positions relative to the aperture for preventing a screw from backing out.

Although the above-described cervical plate connectors as well as others have furthered technological development, none provide enough support to keep the cervical spine in place and hold the vertebra still so they can fuse together for proper healing, while still allowing the spine to articulate naturally. They pursue support, but they do not allow the cervical spine to remain fully mobile as the spine goes from extension to flexion. It would be greatly advantageous to provide cervical plates that allow a spacer mechanism to move along plates connected to vertebrae, as the spine goes from extension to flexion, so that the spine is fully mobile.

### SUMMARY OF THE INVENTION

It is a primary object of the present invention to provide cervical plates with a spacer mechanism that keeps the cervical spine in place and holds the vertebrae still so they can fuse together for proper healing.

It is another object to provide cervical plates with a spacer mechanism whereby the spacer mechanism snaps onto plates so they are not rigidly fixed together.

Yet another object is to provide cervical plates with a spacer mechanism to connect together vertebrae in the cervical spine.

It is yet another object of the present invention to provide cervical plates with a spacer mechanism whereby the spacer mechanism glides along the ridged rails in one direction allowing a degree of flexion in the locking assembly and controlled movement.

Yet another object is to provide an alternative spacer mechanism with slats cut therein to allow for better viewing with X-rays.

Still another object is to provide a locking mechanism that secures the screws into the plates and prevents the screws from backing out of the vertebra.

Another object of the present invention is to provide a connector system and locking mechanism that possess a simple and scalable design.

It is another object of the present invention to provide a connector system and locking mechanism that are fabricated of lightweight materials providing an appropriate degree of flexibility, resiliency, durability, and longevity.

An additional object of the present invention is to provide a connector system and locking mechanism that are inexpensive to manufacture and sell to provide for widespread use.

In accordance with the foregoing objects, the preferred embodiment of the present invention comprises cervical plates and a spacer mechanism. Ridged rails span the entire midline of the plates. The rails run between screw holes, which are cut into the center of one side of each of the two plates. Screws are placed through the screw holes to attach one plate to the superior vertebral body and the other plate to the inferior vertebral body of the cervical spine. A spacer mechanism connects to the ridged rails of plates. The spacer mechanism has a rectangular body with two solid prongs at both the left end and the right end of the spacer mechanism. Both the left and right end of the spacer mechanism have a center prong with apertures in the distal ends, which hook onto the ridged rails when the spacer mechanism is snapped onto the plates. The spacer mechanism moves along the ridged rails until the healing is completed and the two plates are adjacent to each other. The configurations described herein allow the spacer mechanism to have degrees of freedom for easy incorporation with the cervical plates, and also afford durability and resistance to fatigue. As the spine fusion continues to heal, the bones that are healing continue to settle. As the bones are settling the spacer mechanism allows the plates to move and place the fusion under constant pressure. The cervical plates and spacer mechanism disclosed herein allow for the overall variations in spacing between vertebrae of the cervical spine. A locking mechanism locks the screws into the plates of the connector system without detracting from the mobility of the spacer mechanism. The locking mechanism comprises a locking plate having screw holes, and at least one plate cover.

### BRIEF DESCRIPTION OF THE DRAWINGS

Other objects, features, and advantages of the present invention will become more apparent from the following detailed description of the preferred embodiment and certain modifications thereof, in which

FIG. 1 is a front view of the entire cervical plate connector system 1.

FIG. 2 is a front view of the cervical plates 2.

FIG. 3A is a cross-sectional view of the cervical plates 2.

FIG. 3B is a cross-sectional view of the midline of the cervical plates 2.

FIG. 4 is a front view of the spacer mechanism 7.

FIG. 5 is a cross-sectional view of the spacer mechanism 7.

FIG. 6A is a top perspective view of locking mechanism 24.

FIG. 6B is a bottom perspective of locking mechanism 24.

FIG. 7A is a top perspective view of an alternate embodiment of a locking mechanism 25.

FIG. 7B is a bottom perspective view of an alternate embodiment of a locking mechanism 25.

FIG. 8A is a top perspective view of an alternate embodiment of a locking mechanism 37.

FIG. 8B is a bottom perspective view of an alternate embodiment of a locking mechanism 37.

FIG. 8C is a bottom perspective assembled view of an alternate embodiment of a locking mechanism 37.

### DETAILED DESCRIPTION OF THE PREFERRED EMBODIMENTS

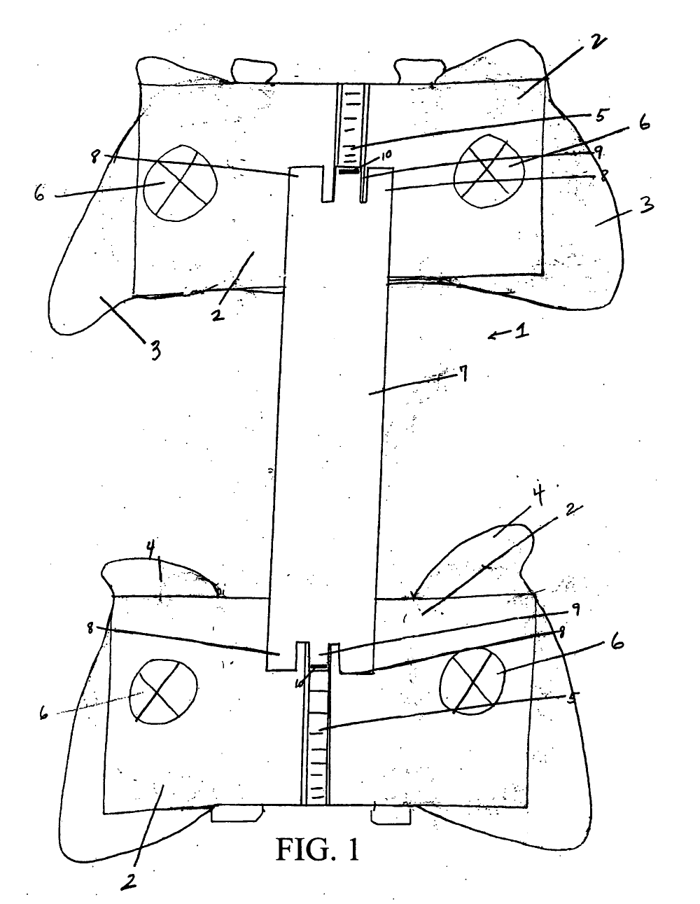

FIG. 1 is a front view of the entire cervical plate connector system 1. The connector system 1 includes at least two plates 2, here shown joining together the superior and inferior vertebral bodies 3, 4 respectively. Plates 2 can be placed up and down the cervical spine in any of its vertebrae. The center of one side of each plate 2 is defined by opposing screw holes 6 for screw-attachment to the superior vertebral body 3 and the inferior vertebral body 4. At least one conventional cervical plate screw is placed through each of the screw holes 6 of the plates 2 into the vertebral bodies 3, 4.

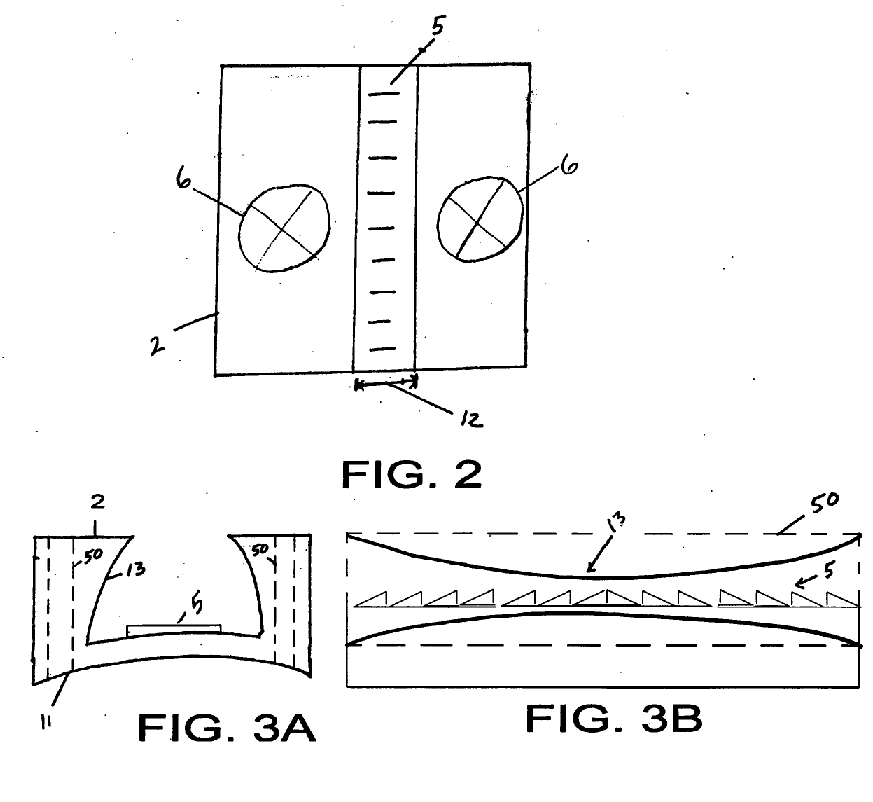

FIGS. 2 and 3A are front and cross-sectional views, respectively, of the plates 2 as in FIG. 1. As best seen in FIG. 3A, the plates 2 may be square or rectangular in shape with a lower convex surface 11 for abutting the vertebral bodies 3, 4. The plates 2 are formed with channels 50 running along the entire midline of the plates 2, bisecting screw holes 6. The sidewalls 13 of the channels 50 are flared inward. As described in more detail below, ridged rails 5 are seated inside the channels 50 along the midline of the plates 2. The ridged rails 5 are elongate members generally comprising an opposing pair of upwardly protruding rails having a saw-tooth track extending there between.

Referring back to FIG. 1, a sliding spacer mechanism 7 connects to the ridged rails 5 of plates 2, thereby joining plates 2 together. Spacer mechanism 7 comprises a flat rectangular body with two solid prongs 8 on opposite sides of a central prong 9 at both the upper and lower ends of the spacer mechanism 7. The spacer mechanism 7 can either be formed as a solid metal body or as a metal body with slats cut therein, which allow straight through, and thus better X-ray viewing. The distal ends of each center prong 9 are defined by apertures 10, the apertures 10 generally conforming to the individual teeth along the saw-tooth track between rails 5 of plates 2 and providing a catch against the teeth of the ridged rails 5. The outer saw-teeth of ridged rails 5 are ramped in two directions and thus the teeth engage the apertures 10 to prevent backing up, while still allowing tightening of the plates 2 (i.e. sliding the plates 2 together).

Thus, the center prong 9 is depressed into the channels 50 of plates 2 and is maintained therein by the inwardly flared sidewalls 13 of the channels 50. The spacer mechanism 7 hooks onto the ridged rails 5 via apertures 10, thereby allowing the spacer mechanism 7 to be secured between opposing plates 2. Prongs 8 rest on the area of the plates 2 directly beyond the ridged rails 5, thereby providing additional lateral stability.

In operation, the spacer mechanism 7 is usually held in tension between opposing plates 2. However, as the superior and inferior vertebral bodies 3, 4 move together, the plates 2 are pushed completely adjacent to each other. Because the spacer mechanism 7 is able to snap onto and move along the ridged rails 5 of the plates 2, there is a degree of flexion in the locking assembly to accommodate the fusion. The center prongs 9 of the spacer mechanism 7 move along the rails 5 as the superior and inferior vertebral bodies 3, 4 move together, and yet the center prongs 9 may be depressed at any point to incrementally engage the apertures 10 with the outer saw-tooth cross section of ridged rails 5, thereby locking the spacer mechanism 7 to the plates 2 in a manner allowing freedom of movement, and yet to prevent backing up. Thus, the center prongs 9 are deformable.

FIG. 3B is a cross-sectional view of the midline of the plates 2. These Figures illustrate that the ridged rails 5 running along the midline, are submerged in channels 50 of the plates 2. The width 12 of the ridged rails 5 may vary so long as they leave room for the screw holes 6.

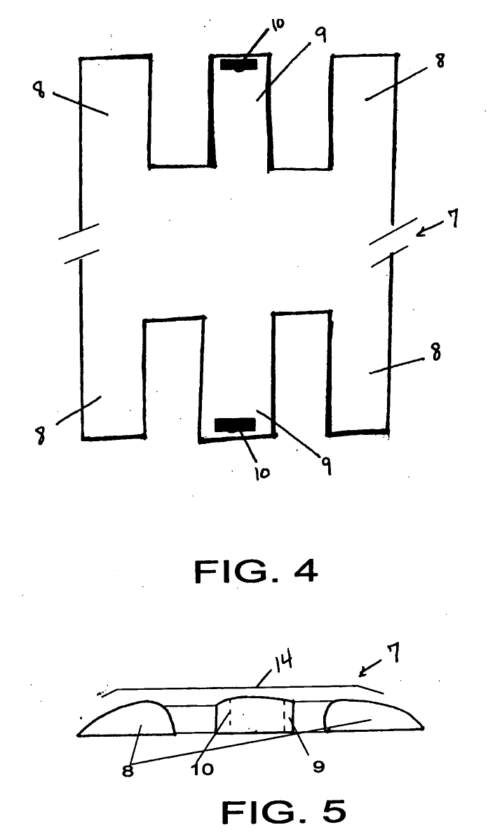

FIGS. 4 and 5 are front and cross-sectional views of the spacer mechanism 7. The spacer mechanism 7 has a contoured (concave) upper surface 14.

With combined reference to FIGS. 2, 3A, 3B, 4, 5, the radius of curvature of the convex surface 11 of the plates 2 exceeds the radius of curvature of the concave surface 14 of the spacer mechanism 7. This allows center prong 9 to snap into the plates 2 for a close yet flexible fit. Also, the spacer mechanism 7 has the ability to move as the cervical spine goes from extension to flexion because the assemblies described herein are not fixed. Thus the present invention allows the cervical spine to remain fully mobile.

The present invention also comprises a locking mechanism that locks the screws into the plates 2 of the connector system 1 without detracting from the mobility of the spacer mechanism 7 described above. This may be accomplished with a variety of alternative and equally well-suited configurations, three of which are described in detail herein.

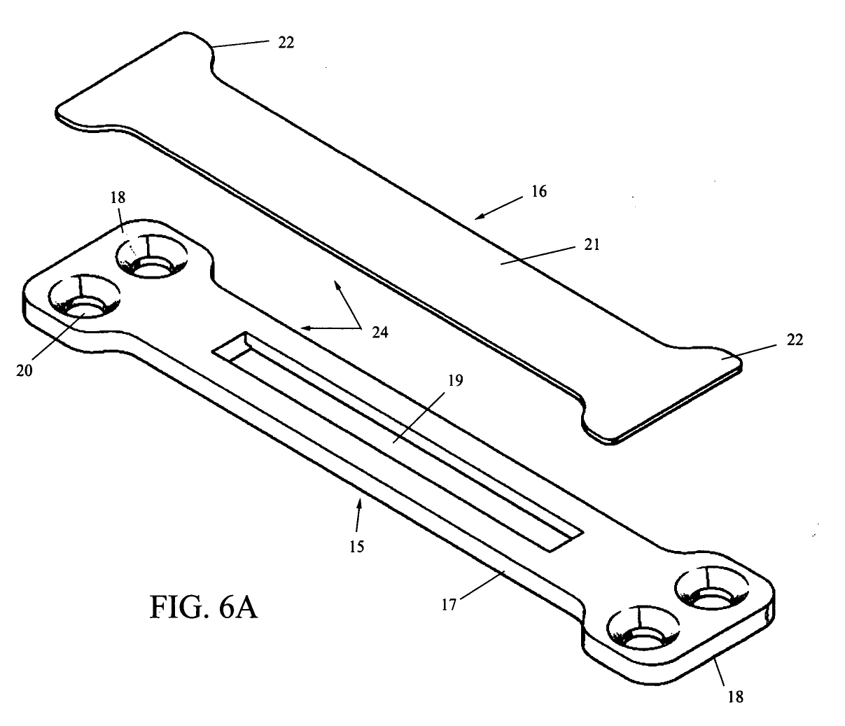

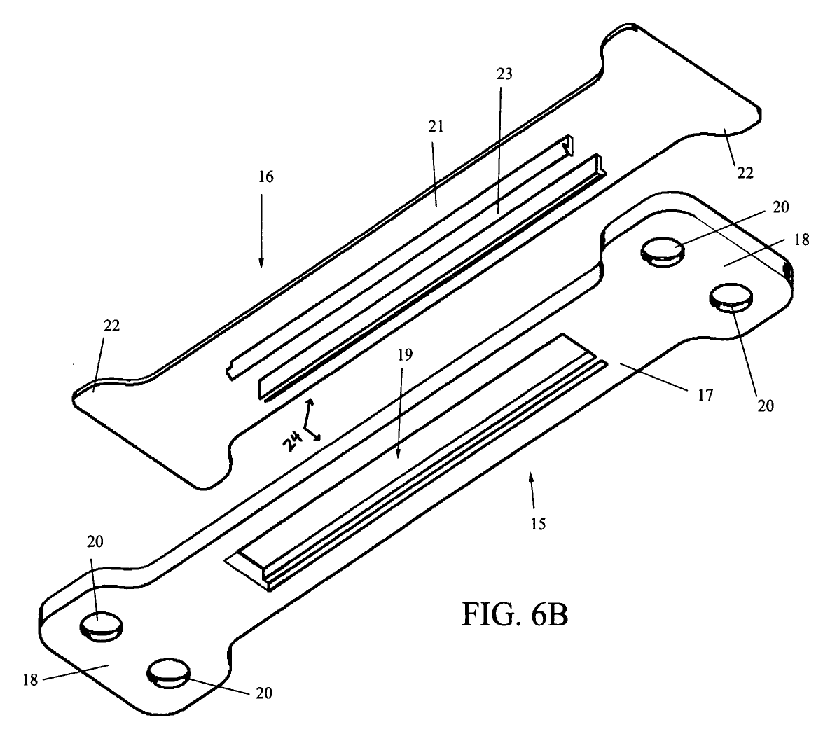

FIGS. 6A and 6B are top perspective and bottom perspective views, respectively of a preferred embodiment of a locking mechanism 24 which comprises a locking plate 15 and cover 16. The locking plate 15 is a long rectangular body 17 with flared ends 18. The rectangular body 17 has a centrally defined slat 19. Each of the flared ends 18 has screw holes 20 (two are illustrated) drilled therethrough. The cover 16 is a long rectangular body 21 with flared ends 22. The topside of the cover 16 is flat. The underside of the cover 16 has a central rectangular tab 23 protruding from the rectangular body 21.

In use, the surgeon positions the plates 2 of the connector system 1 over the superior and inferior vertebral bodies 3, 4 and then horizontally places the locking plate 15 between the screw holes 6 in the plates 2 of the connector system 1. The surgeon then inserts screws through the screw holes 20 in the locking plate 15 and then through the holes 6 in the plates 2 of the connector system 1. The rectangular tab 23 of the cover 16 snap fits through the slat 19 of the locking plate 15. After the locking plate 15 is affixed to the plates 2, it sits above the spacer mechanism 7 and does not interfere with the motion of the spacer mechanism 7. The flared ends 22 of the cover 16 overlie the screw heads protruding through the screw holes 20, thereby ensuring that the screws do not back out of the vertebra. The cover 16 is long enough to overlie the screw heads, but it does not extend the full length of the locking plate 15.

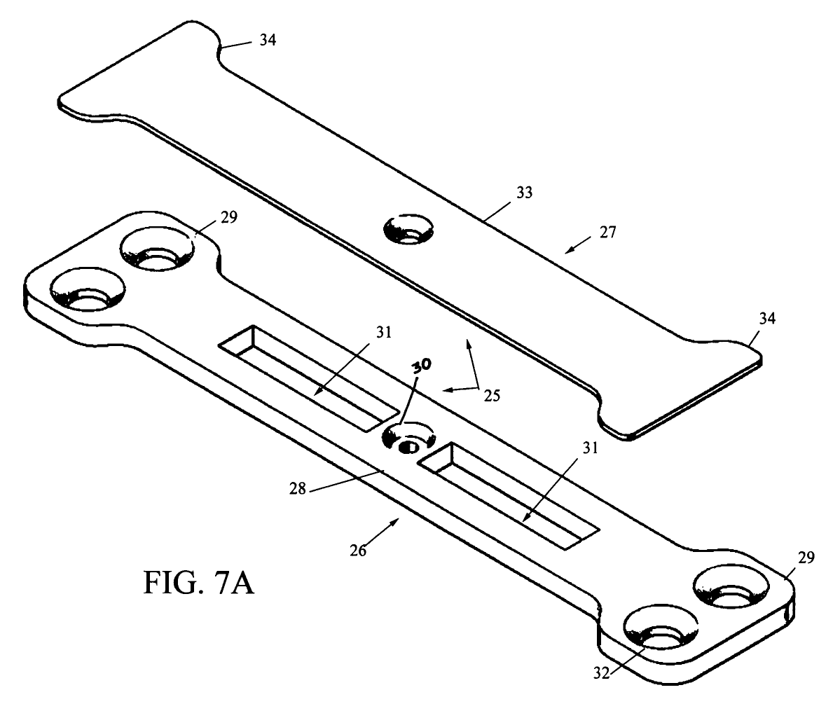

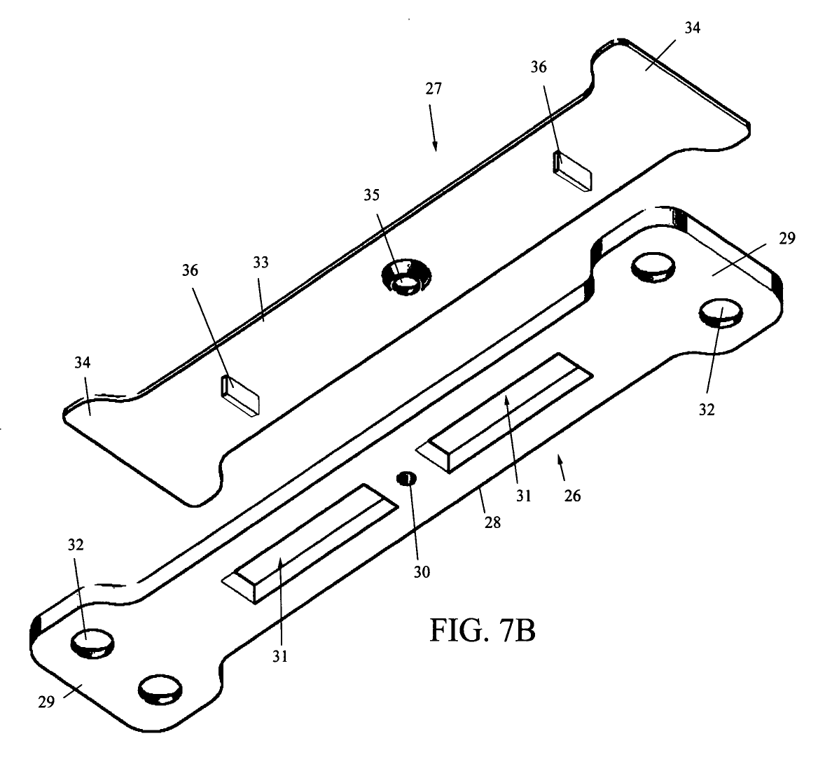

FIGS. 7A and 7B are top perspective and bottom perspective views, respectively of an alternate embodiment of a locking mechanism 25. The locking mechanism 25 comprises a locking plate 26 and cover 27. The locking plate 26 is a long rectangular body 28 with flared ends 29. The rectangular body 28 has a centrally defined recess 30, with slats 31 defined on each side of the recess 30. Each flared end 29 has screw holes 32 (two are illustrated) drilled into it. The cover 27 is a long rectangular body 33 with flared ends 34. The topside of the cover 27 is flat. The underside of the cover 27 has a centrally defined protuberance 35 that narrows in diameter as it extends downward. Two stops 36 protrude from the underside of the cover 27, one near each end of the rectangular body 33. The protuberance 35 and recess 30 are configured such that the protuberance 35 twist-locks into the recess 30.

In use, the surgeon positions the plates 2 of the connector system 1 over the superior and inferior vertebral bodies 3, 4 and then horizontally places the locking plate 26 between the screw holes 6 in the plates 2 of the connector system 1. The surgeon then inserts screws through the screw holes 32 in the locking plate 26 and then through the holes 6 in the plates 2 of the connector system 1. The protuberance 35 of the cover 27 twist-locks into the recess 30 of the locking plate 26, and each stop 36 fits through a corresponding slat 31, for securing the cover 27 and locking plate 26 in place. After the locking plate 26 is affixed to the plates 2, it sits above the spacer mechanism 7 and does not interfere with the motion of the spacer mechanism 7. The flared ends 34 of the cover 27 overlie the screw heads protruding through the screw holes 32, thereby ensuring that the screws do not back out of the vertebra. The cover 27 is long enough to overlie the screw heads, but it does not extend the full length of the locking plate 26.

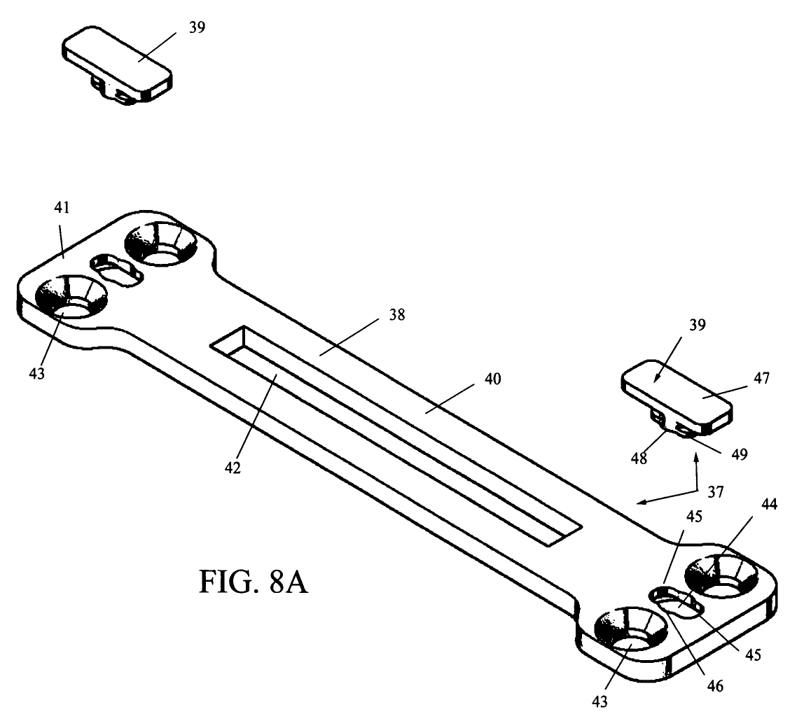

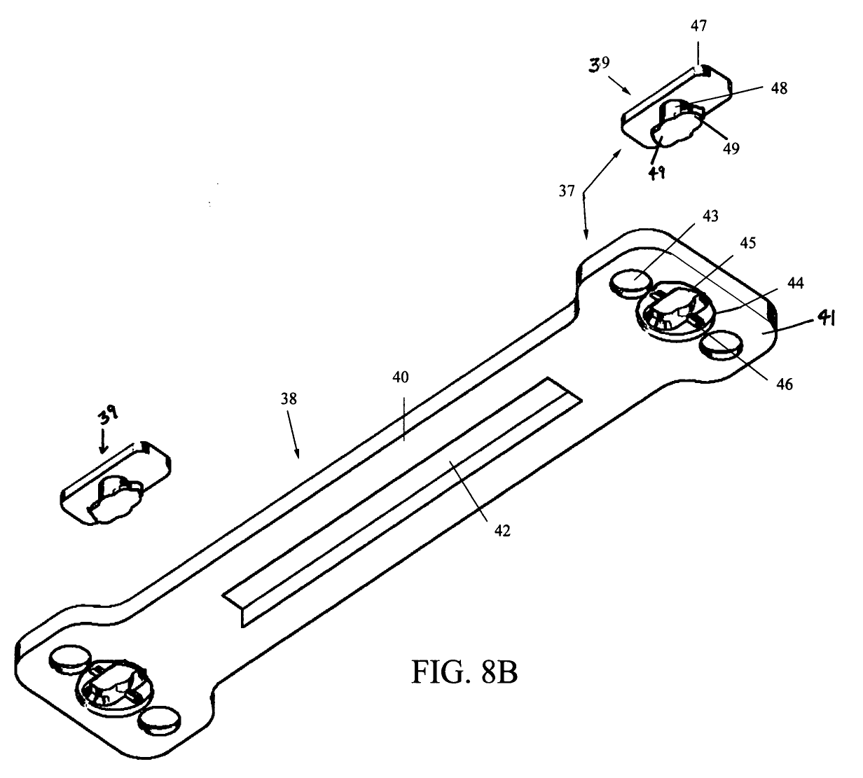

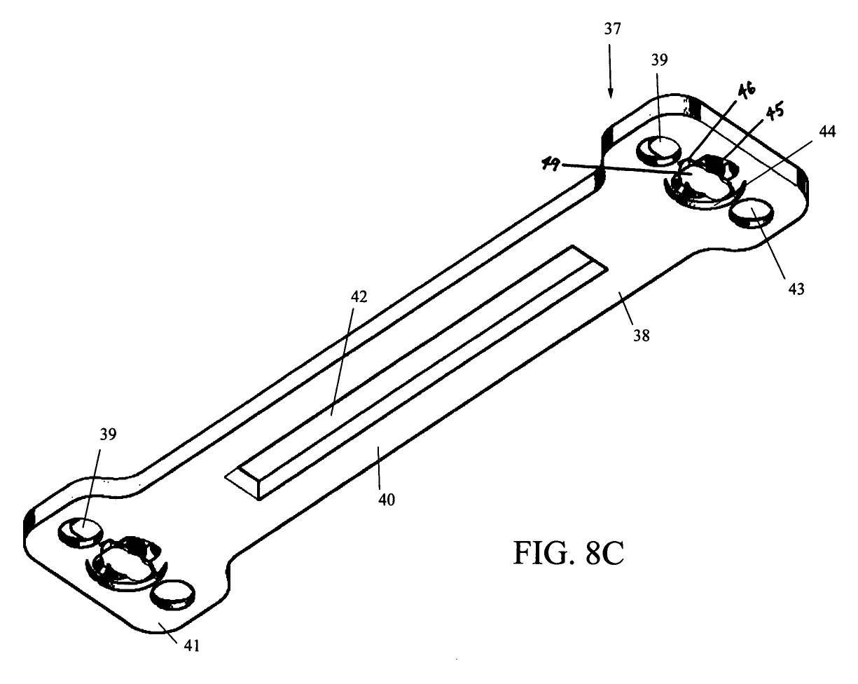

FIGS. 8A-8C are top perspective, bottom perspective, and bottom perspective assembled views, respectively of yet another alternate embodiment of a locking mechanism 37, which comprises a locking plate 38 and two covers 39. The locking plate 38 is a long rectangular body 40 with flared ends 41. The rectangular body 40 has a centrally defined slat 42. Each flared end 41 has screw holes 43 (two are illustrated) drilled into it. Apertures 44 bisect the screw holes 43 in the flared ends 41. Each aperture 44 has a lip 45 interrupted with keyhole slots 46. The covers 39 each generally comprise a short rectangular body 47. The topside of each cover 39 is flat. The underside of the cover 39 has a centrally protruding peg 48. The peg 48 has two opposing tabs 49 extending laterally around it proximate its distal end. The tabs 49 are configured for downward insertion into the keyhole slots 46 in the apertures 44. The pegs 48 are inserted into the slots 46 by a simple push-and-twist operation which locks the tabs 49 therein, preventing inadvertent withdrawal.

In use, the surgeon positions the plates 2 of the connector system 1 over the superior and inferior vertebral bodies 3, 4 and then horizontally places the locking plate 38 between the screw holes 6 in the plates 2 of the connector system 1. The surgeon then inserts screws through the screw holes 43 in the locking plate 38 and then through the holes 6 in the plates 2 of the connector system 1. The pegs 48 of the covers 39 twist-lock into the slots 46 of the apertures 44 of the locking plate 38. After the locking plate 38 is affixed to the plates 2, it sits above the spacer mechanism 7 and does not interfere with the motion of the spacer mechanism 7. The ends of the covers 39 overlie the screw heads protruding through the screw holes 43, thereby ensuring that the screws do not back out of the vertebra. To additionally secure the screws, cover 16 can snap fit into the slat 42 of the locking plate 38. The flared ends 22 of the cover 16 would overlie the ends of the short covers 39 and doubly enforce the screws in the vertebra to ensure that they do not back out.

The plates in FIGS. 1, 2 and 3A show one screw hole 6 on each side of plate 2, and thus the corresponding locking mechanism would have locking plates with one screw hole drilled into each of the locking plates. One skilled in the art will recognize that additional screw holes 6 can be drilled into the plates 2 to accommodate additional screws for added strength and stability.

The connector system 1 and locking mechanisms 24, 25, 37 possess a simple and scalable design. Additionally, the connector system 1 and locking mechanisms 24, 25, 37 are fabricated of lightweight materials providing an appropriate degree of flexibility, resiliency, durability, and longevity. Also, the connector system 1 and locking mechanisms 24, 25, 37 are inexpensive to manufacture and sell, to provide for widespread use.

All of the above components can be made of any suitable surgical materials, such as, for example, stainless steel or titanium.

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth in the appended claims.

I claim:

1. A cervical plate connector system, comprising:
a plurality of plates adapted to be secured to vertebral bodies in a spinal column, each of said plurality of plates including a plurality of screw holes;
a corresponding plurality of screws for insertion into said screw holes for securing said plates to said vertebral bodies; and
a spacer mechanism connecting said plates together.

2. A cervical plate connector system, comprising:
a top plate adapted to be secured to upper vertebra in a spinal column, and a bottom plate adapted to be secured to lower vertebra in a spinal column, both of said top plate and bottom plate including a convex surface for abutting the upper and lower vertebra, respectively, a pair of opposing screw holes through said plates, and a channel running along a midline of said plates between said opposing screw holes;
a pair of ridged rails each recessed inside a corresponding channel of said top plate and bottom plate;
a plurality of screws for insertion into said screw holes and attachment of said plates to said upper and lower vertebra; and
a spacer mechanism comprising an elongate member adapted for slidable insertion into the ridged rails in said top and bottom plates for securing said ridged rails and yet permitting relative axial movement of said top and bottom plates.

3. A cervical plate connector system according to claim 2, wherein each of said ridged rails further comprises an opposing pair of upwardly protruding rails having a channel there between.

4. A cervical plate connector system according to claim 3, wherein each of said ridged rails further comprises sawtooth track extending along the channel between said protruding rails.

5. A cervical plate connector system according to claim 4, wherein said spacer mechanism comprises an elongate member having two distal ends each formed with a central prong for engagement with the teeth of said sawtooth track to permit one-way sliding.

6. A cervical plate connector system according to claim 5, wherein said spacer mechanism comprises an elongate member having two distal ends each formed with two solid end prongs and a recessed central prong.

7. A cervical plate connector system according to claim 2, wherein said system is comprised of a biocompatible material.

8. A cervical plate connector system according to claim 3, wherein each of said ridged rails further comprises a channel formed with inwardly flared sidewalls.

9. A cervical plate connector system according to claim 5, wherein said central prong is defined by an aperture for capturing the teeth of the sawtooth track in said ridged rails to secure said spacer mechanism to said plates.

10. A cervical plate connector system according to claim 9, wherein said spacer mechanism comprises a solid metal body.

11. A cervical plate connector system according to claim 1, wherein said spacer mechanism is defined by elongate open slats to allow X-rays to pass through.

12. A cervical plate connector system according to claim 2, further comprising a locking mechanism to prevent withdrawal of said screws from vertebral bodies.

13. A cervical plate connector system according to claim 12, wherein said locking mechanism further comprises a locking plate fixedly connectable to said plates, and a cover fixedly connectable to said locking plate.

14. A cervical plate connector system according to claim 13, wherein said locking plate comprises a substantially rectangular body having a centrally defined slat and flared ends formed with a plurality of screw holes.

15. A cervical plate connector system according to claim 13, wherein said cover comprises a substantially rectangular body having a flat topside, an underside comprising a central rectangular tab for snap-fitting into said slat, and flared ends that overlie said screw holes in said locking plate, thereby preventing said screws from backing out.

16. A cervical plate connector system according to claim 13, wherein said locking plate comprises a substantially rectangular body having a centrally defined recess, a plurality of slats on each side of said recess, and flared ends having a plurality of screw holes.

17. A cervical plate connector system according to claim 13, wherein said cover comprises a substantially rectangular body having a flat topside, an underside comprising a plurality of tabs for passing through said slots, and a centrally defined protuberance for twist-locking into said recess, and flared ends that overlie said screw holes in said locking plate, thereby preventing said screws from backing out.

18. A cervical plate connector system according to claim 13, wherein said locking mechanism further comprises a locking plate fixedly connectable to said top and bottom plates, and two covers fixedly connectable to said locking plate.

19. A cervical plate connector system according to claim 13, wherein said locking plate further comprises a substantially rectangular body having a centrally defined slat, flared ends having a plurality of screw holes, and two apertures having lips interrupted with keyhole slots, wherein each of said apertures bisects said screw holes in said flared ends.

20. A cervical plate connector system according to claim 18, wherein said each of said covers further comprises a substantially rectangular body further comprising a topside, wherein said topside is flat; an underside comprising a central peg having a plurality of tabs for snap-and-twist insertion into said keyhole slots of aperture; and flared ends that overlie said screw holes in said locking plate, thereby preventing said screws from backing out.

21. A cervical plate connector system according to claim 13, further comprising a cover having a substantially rectangular body having a flat topside, an underside comprising a central rectangular tab for snap-fitting into said slat, and flared ends that overlie said flared ends of said short covers, thereby preventing said screws from backing out.
